(19) United States
(12) Patent Application Publication
(10) Pub. No.: US 2010/0037369 A1
(43) Pub. Date: Feb. 18, 2010

(54) MUSCLE SUPPORT GARMENT AND METHOD

(76) Inventor: Andreas B. Reichert, Washington, DC (US)

Correspondence Address: MERCHANT & GOULD PC, P.O. BOX 2903, MINNEAPOLIS, MN, 55402-0903, US

(21) Appl. No.: 12/541,289

(22) Filed: Aug. 14, 2009

Related U.S. Application Data
(60) Provisional application No. 61/089,580, filed on Aug. 18, 2008.

(51) Int. Cl. A41D 1/08; A61F 13/06

(52) U.S. Cl. 2/228; 602/63

(57) ABSTRACT

A muscle support garment and method for applying compressive forces to muscles within a treatment area are disclosed. A band of stretchable wrap material is secured to the garment and is wound in an overlapping spiral manner on the inside of the garment, encircling the treatment area. The band of wrap material is longitudinally adjustable to vary its tension and the compressive forces applied, from external of the garment. Overlapping loop retainer structures secured inside the garment slidably entrain and retain the wrap material in an overlapping manner. The garment may include an inner liner.

### CROSS REFERENCE TO RELATED APPLICATIONS

The complete disclosure of U.S. Provisional Application 61/089,580, filed Aug. 18, 2008 is incorporated herein by reference. This application claims of priority to U.S. Provisional Application 61/089,580.

### FIELD OF THE INVENTION

This invention relates generally to therapeutic garments and more specifically to an improved apparatus and method for supporting muscles, and particularly hamstring and thigh muscles to help prevent muscle injury and for providing muscle support during healing of injured muscles.

### BACKGROUND

There are a number of muscles that can benefit from the application of compressive force to the muscle, either during exercise or use of such muscles, or during therapeutic or rehabilitative treatment or conditioning of such muscles. While this invention applies to the application of such compressive forces to muscles in general, that muscle group to which the inventive preferred embodiment will be directed will be for treatment of the “hamstring” muscles. It will be understood that the disclosure herein is illustrative of one preferred embodiment of the invention, and is not to be read or interpreted in a limiting manner to apply only to structures designed specifically for the treatment of hamstring muscles. It will also be understood that the invention, by providing compressive forces to muscles and body tissue may also, have beneficial effects in the treatment of tendons and ligaments, by for example, properly aligning or restricting movement of muscle groups in manners that reduce injury or stress to tendons or ligaments.

The so-called hamstring muscles generally comprise three key large muscle groups that span the thigh and cross the hip and knee. They are the semimembranosus, the semitendenosis and the long and short heads of the biceps femoris muscle groups. The hamstring muscles cooperate to actively bend or flex the knee and act to straighten or extend the hip, such as in the motion of moving the thigh in a backwards direction. These large muscles are typically not very active during normal walking activities, but are particularly active and important during power activities, such as running, jumping or climbing, or for activities that require a person to make frequent starts and stops. While all sports require start/stop action to some degree, such action is particularly prevalent in sports such as football, soccer, basketball and tennis. Sedentary individuals can generally function well with weak deconditioned hamstring muscles. However, athletes or individuals who are very physically active depend on healthy well-conditioned hamstring muscles.

Hamstring injuries usually occur as a result of running or jumping, particularly when one of the person's legs is “lunging forward”. Sometimes a “pop” is heard or felt by the injured person. Tears or strains most often occur at the middle of the back of the thigh where the muscle joins its tendon or at the origin of a hamstring muscle at the base of the buttocks (at the ischium). Injuries to the hamstring group of muscles can range from minor strains to major ruptures. Minor strains are classified as Grade I tears; whereas, complete ruptures are classified as Grade III tears. Partial ruptures are classified as Grade II tears. Injury to the hamstring muscles can be minimized by strengthening the hamstring muscles and by maximizing muscle flexibility by regular stretching programs and proper warm-up stretching regimens just prior to entering into strenuous athletic activity. The present invention provides a garment that provides both strength and flexibility in properly supporting the hamstring muscles during such injury preventative programs and which also supports the hamstring muscles during strenuous physical activities that task the hamstring muscles.

Most hamstring injuries are treated without surgery. The goal of treatment is to restore muscle function and to prevent scar formation. Initial treatment generally consists of rest, ice, compression and elevation, often referred to as R.I.C.E. When pain reduction permits, it is important to begin a program of stretching and range of motion exercises. Reinjury which prolongs recovery, and which increases the risk of permanent damage, can occur by premature return to the sport or activity. The present invention provides an apparatus and method that enables proper support to and flexibility of the hamstring muscles during the recovery program.

A number of hamstring muscle support mechanisms are known in the art. The most basic technique is to apply a compression wrap directly to the thigh, which extends from the upper thigh, down to or just above the knee area, in a continuous, winding spiral overlapping manner. The leading end of the wrap is generally unsecured and held in place against the thigh by the compressive force of overlying wrap layers, and the trailing end of the wrap is secured to itself in well-known manner, similar to that used to secure Ace bandage-type wraps. Such wrap underlies and is unattached to any overlying short or pant garment and is of an elastic stretchable material.

Other techniques and specially configured mechanisms for providing compressive force to the thigh and or knee area are known. Broad compressive support to the skin and muscles of the abdomen, buttocks and thighs is provided by a compression short garment, extending from the wearer's waist to just above the knee and typically made from a combination of nylon and elastomeric materials. Other support devices designed to more specifically apply compressive pressure to the hamstring muscles are configured to wrap around the wearer's thigh and upper leg and extend downwardly to just above the knee. A simple such mechanism comprises a single continuous tube or stocking type of elastic compression material that is pulled up over a person's foot and calf and positioned to overlie the thigh. Such devices generally need to be manufactured in different sizes to accommodate thighs of different diameters and do not provide for adjustment of the applied compressive force. Another mechanism uses a closable wrap of compressible material that is placed in surrounding manner around the wearer's thigh and secured to itself along one longitudinal edge by an adjustable fastener, such as a hook and loop (Velcro®-type) fastener. A variation of the above mechanism uses a tubular sleeve of neoprene material sized to surround the thigh and has various types of external tightening straps or fasteners for adjustably applying compressive forces to the thigh muscles. Such thigh supports may include insertable and/or adjustable pads, or provisions for hot or cold pack treatments.

Other known leg muscle supports include a thigh-to-ankle strap/brace structure responsive to knee bending motion, and a neoprene-type short material configured to apply both heat and compressive forces to both legs of the wearer. While each of such known devices provide some measure of therapeutic support to the hamstring muscles, they do not generally simultaneously provide the needed muscle support, flexibility of use, ease of adjustability or comfort that make them both therapeutically useful and user friendly.

### SUMMARY OF THE INVENTION

According to one aspect of the invention, there is provided an improved hamstring muscle and thigh support garment that applies overlapping compression forces to the three key hamstring muscles in a manner that minimizes slippage of the support mechanism relative to the thigh while providing support and regenerative benefits for sore, tight or weak hamstring muscles or for hamstring muscle sprains. The support garment of the present invention carries an adjustable spirally overlapping elastive compression wrap material that is applied directly to the thigh of the wearer from inside of the garment, or which is separated from the thigh by a thin liner material that reduces skin irritation and chaffing and improve wearer comfort. The wrap material is, however, firmly held in the desired location overlying the thigh by the garment in a manner that minimizes slippage or bunching of the wrap material or separation of the overlapping wrap bands. The compressive force applied by the wrap material is readily adjustable by the wearer through simple wrap tension adjustment motion, achieved from a position external of the garment. The garment can be configured to be used as an outerwear garment, or as an inner garment to be worn under other sports garments or athletic equipment. The wrap adjustability feature is generally readily accessible to a wearer, even when the support garment is used as an inner garment that underlies outer clothing or equipment.

The invention need not be limited, however, to the treatment or support of hamstring muscles, but can apply to the treatment of muscles in other treatment portions of a person's body. Therefore, according to another aspect of the invention, there is disclosed a muscle support garment having: an outer garment configured in part to encircle a treatment portion of a person's body; an elongated band of wrap material with first and second ends, wherein at least one of the ends is securable to the outer garment; a retainer assembly configured to operatively mount a substantial portion of the band of wrap material inside of the outer garment, in spiral overlapping manner encircling the body treatment portion; and adjustment apparatus operatively connected to said band of wrap material and configured to provide length adjustment tensioning of the wrap material about the body treatment portion, from external of the outer garment. According to a further aspect of the invention, the adjustment apparatus comprises in part, a portion of the band of wrap material extending externally of the outer garment that can be grasped and pulled longitudinally of the band to exert a desired tension to the band of wrap material.

According to yet another aspect of the invention, the support garment comprises a compression pant having at least one leg portion, an elongate wrap band, and a wrap retaining apparatus configured to operatively connect the elongate wrap band to the compression pant and to align the elongate wrap band along an inner circumferential surface of a pant leg portion of the compression pant, in continuous overlapping spiraling manner to encircle the thigh portion of a wearer of the garment. The compression pant could be of a light-weight condensation polymer and elastive material, or could comprise a heavier material such as neoprene, or a combination of both. According to a preferred construction of the support garment, the compression pant also includes an upper torso surrounding portion comprising a light-weight condensation polymer and elastive material that provides maximum comfort to the wearer; whereas at least a portion of the leg portions of the compression pant could comprise heavier material suitable for anchoring the wrap retaining apparatus.

According to a preferred configuration of the invention, the wrap retaining apparatus comprises a plurality of loop structures configured for attachment to the compression pant leg portions along an inner surface thereof for addressing the thigh of the wearer. The elongate wrap band is sized and configured to slide within loops of the loop structure in an overlapping spiraling configuration that enables longitudinal adjustment to selectively apply tension to the band, thus providing adjustable compressive radial forces to the thigh of the wearer. According to one aspect of the invention, at least one leg portion of the compression pant defines an opening therethrough, through which one end of the wrap band is entrained. The one end of the wrap band is adjustably detachably anchorable to a fastener member secured to an outer surface of the compression pant leg portion. The wearer can rapidly access the wrap band and adjust its longitudinal tension and thus the radial compressive force applied by the wrap to the wearer's leg by grasping and adjusting the position of the one end of the wrap band relative to its fastener member on the outside of the leg portion surface.

According to a preferred configuration of the support garment, the material for anchoring the wrap retaining loop structures comprises neoprene material. A preferred material for the elongate wrap band comprises a four-way stretchable material such as spandex. A preferred material according to one aspect of the invention for the retaining loop structures is a heavier nylon webbing material.

According to another aspect of the invention there is provided a method of applying a compressive wrap to a thigh portion of a person comprising the steps of: providing a compression pant having at least one leg portion for surrounding the thigh portion; securing a longitudinally overlapping loop arrangement to the inside of the leg portion; entraining an elongate wrap band through a plurality of loops of the loop arrangement such that the wrap band encircles the wearer's thigh portion in overlapping spiraling manner; longitudinally pulling on the wrap band to apply radial compressive force to the thigh portion; and securing the ends of the wrap band to the leg portion to maintain a desired compressive force against the thigh portion.

The desired compressive force applied can be adjusted by: detaching one end of the wrap band from the leg portion; causing the wrap band to longitudinally move and slide within the loop arrangement to change the compressive force applied to the thigh portion; and refastening the one end of the wrap band to the leg portion to maintain the changed compressive force.

As stated above, while the invention is particularly applicable to the treatment of hamstring muscles, its principles apply to the treatment of other muscles as well. The invention is particularly relevant to the treatment of muscles in treatment portions of a person's body that can be encircled by the outer garment such that the band of wrap material can be configured to operatively encircle and apply adjustable compressive forces to such muscles in the body treatment portion.

It will be appreciated that materials other than those specifically disclosed herein could be used. Further, while various dimensions and properties for materials and components described with respect to preferred embodiments of the invention are recited, such dimensions and properties can be varied within the spirit and scope of this invention.

These and other aspects of the invention and design variations will be apparent to those skilled in the art in view of the below descriptions of preferred embodiments of the invention.

### BRIEF DESCRIPTION OF THE DRAWINGS

Referring to the drawings, wherein like numerals represent like parts throughout the several views:

FIG. 1 is a frontal diagrammatic view of a first embodiment of a muscle support garment of this invention, illustrated as it would appear when worn by a person for supporting hamstring muscles of the person;

FIG. 2 is a back perspective view of the muscle support garment of FIG. 1, viewed with a portion of the outer material of the right leg broken away;

FIG. 3 is a diagrammatic cross-sectional view of the right leg portion of the muscle support garment of FIG. 2, generally taken along the Line 3-3 of FIG. 2;

FIG. 4 is a diagrammatic view of the support loop structures for the stretchable wrap strap material of the left leg portion of the muscle support garment of FIG. 1, viewed toward the front of the left leg and illustrating in dashed lines how the wrap material operatively winds through and is entrained by the supporting loops; and

FIG. 5 is an enlarged fragmentary view of a portion of the inner left leg of the muscle support garment of FIG. 1.

### DETAILED DESCRIPTION OF THE PREFERRED EMBODIMENT

The below described preferred embodiment of the invention provides a light-weight easy to use garment that selectively provides hamstring muscle support for both legs of the wearer and which offers rapid compressive force adjustment for each leg by a simple pulling motion from the outside of the garment. In the preferred embodiment shown, the compressive force adjustment feature is accessible along a front portion of each leg of the garment, just above the knee, so that the adjustment can be rapidly made even when a person is wearing other garments or sportswear clothing over the hamstring support garment. While the present invention will be disclosed in association with a shorts-type garment whose lower edge terminates near the wearer's knee, the principles of the invention as applicable to hamstring and other leg muscle support, could be incorporated as well into a full length pants garment. A full length pants garment, might include multiple compressive wrap areas along the leg or torso of the wearer, to address those muscle groups of, for example, the leg, knee, or abdomen areas being treated.

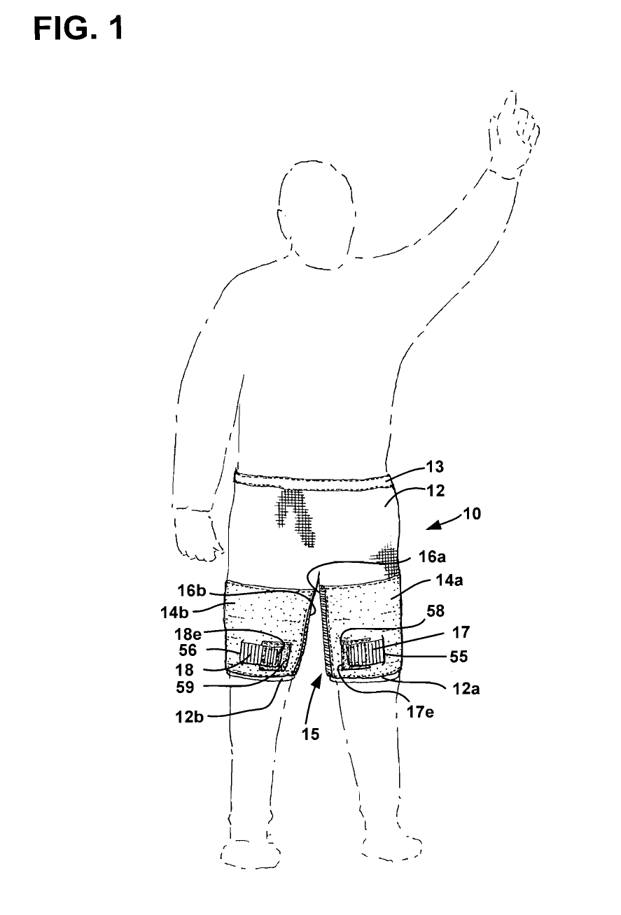

A first embodiment of a hamstring support garment incorporating the principles of the invention is illustrated at 10 in FIG. 1. An underlying compression pant 12 of stretchable material is sized to lie between the waist and downwardly to just above the knee of the wearer. Such compression pants are well-known in the art and are commonly worn by athletes or others engaging in physical activities, below outer sportswear or clothing. In the preferred embodiment, the compression short material is comprised of about 85% condensation polymer material and about 15% elastive materials. In the preferred embodiment, the condensation polymer material is preferably nylon and the elastive material is a spandex, elastine or elastic material. The compression pant typically has an upper band 13 of elastic material or alternatively could comprise a continuous loop encircling the torso at the waist, which is configured to hold a drawstring that can be adjustably tightened around the user's waist. It will be appreciated that materials other than nylon could be used. A material that provides wicking of moisture from the wearer's skin is preferred. Also, the percentage of elastive material in the compression pant can vary from the 15% noted above. Preferably, the compression pant would have from about 5% to 20% of elastive material. Other materials that could, for example, be used in place of nylon include polyesters, polycarbonates and thin (less than about 0.5 mm thick) neoprene. The material of the compression pant provides a gentle uniform compressive supporting force to the engaged skin and underlying muscle portions of the wearer, and enables outerwear garments or sporting equipment to slide over the compressive pant material to minimize irritation or chaffing of the underlying skin. The compression pant forms an inner shell for the hamstring support garment and positions the compressive wrap feature of the invention (discussed below) in place overlying the thigh portions of the body. The compressive pant material also prevents direct contact of the loop and wrap materials of the invention with the skin of the wearer, thus minimizing skin chaffing by such components.

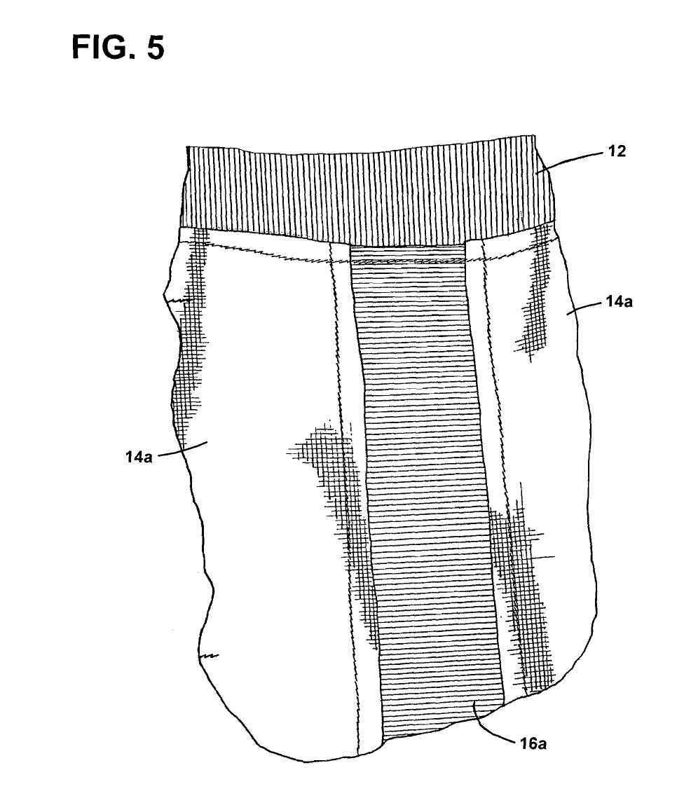

Left and right support sleeves of an elastomer such as neoprene material 14a, 14b are sewn along their upper and lower ends to the underlying inner shell material of the compression pants to the left and right leg portions 12a and 12b respectively thereof. A thick spandex material could be used as alternative to neoprene. The neoprene sleeves extend longitudinally generally from the crotch area to the lower leg edges of the compression pant 12 to generally surround the thighs of the wearer. In the preferred embodiment, the support sleeves 14a and 14b are attached to the inner shell 12 material only along their upper and lower ends, thereby providing generally free movement of the support loop and wrap material (hereinafter described) between the inner surfaces of the neoprene sleeves and the outer surfaces of the compression pant material. In the preferred embodiment, each of the neoprene sleeves 14a, 14b is longitudinally split or separated along the inner thigh area as shown at 15 in FIGS. 1 and 5, and incorporates an elongate expansion strip of material 16a and 16b respectively sewn to and between the opposing longitudinally separated edges of the neoprene material, to enable the neoprene sleeves to expand with the underlying inner shell material of the compression pant to accommodate various thigh circumferences and shapes. The expansion strips 16 also enable those applied compressive forces of the garment 10 to be administered primarily by the overlapping wrap material, and not by the neoprene sleeve material itself.

The neoprene sleeve material provides positioning and anchoring support for the elastic wrap and wraps retaining loop portions of the invention. The neoprene material needs to be sufficiently thick to provide adequate anchoring support for the loop structures and to prevent the wrap material from longitudinally moving or bunching upon the thigh, but also should be thin enough to provide comfort to the wearer. In the preferred embodiment, the neoprene material is about 1.5 mm thick. However, the thickness could preferably vary from about 0.2 mm to about 5.0 mm in thickness.

It is also possible to construct the entire compression pant from a combination of neoprene material and elastive material, and to eliminate the outer thigh sleeves. However, such construction is not as comfortable to the wearer as compared to the preferred construction, wherein the upper portion of the compression pant is formed from polyester and elastive materials.

It has been found that an effective support treatment for hamstring muscles, either as an injury prevention measure or during hamstring injury recovery programs, is to wrap the thigh and underlying hamstring muscles with an elastic wrap material, downwardly spiraling in overlapping manner along the length of the thigh, from the buttocks area down to just above the knee. Such wrap has been found to be most effective when applied directly to the skin as opposed to overlying a garment to which it is unattached which causes the wrap to ride up or longitudinally move relative to the thigh. However, when applied directly to the skin, the wrap can cause irritation to the skin and can, with athletic movement of the thigh, have a tendency to bunch up over time. The present invention addresses these issues of the prior art, while providing an effective overlapping/spiraling compressive and adjustable wrap to the hamstring muscles.

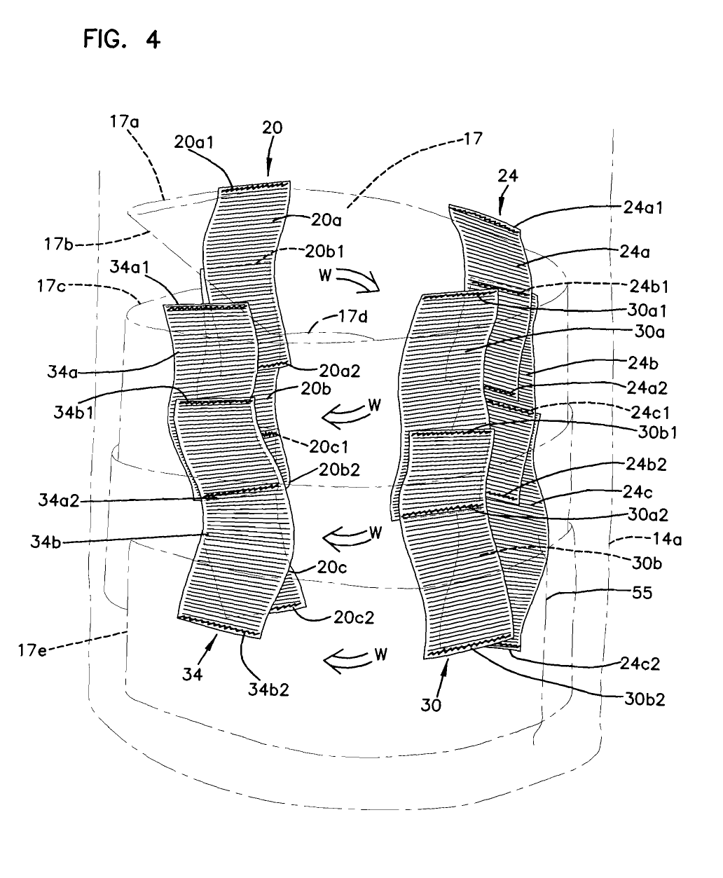

Proper longitudinal positioning of the wrap material in a downwardly spiraling and overlapping manner is provided by a plurality of web support loop structures. FIG. 4 is a diagrammatic view illustrating an example of a preferred positioning of four such support loop structures as they would appear secured to and around the inner circumferential surface of the neoprene sleeve 14a overlying the left leg portion 12a of the compressive pant 12. The loop structures of FIG. 4 appear as viewed from the upper front of the left leg of FIG. 1. The neoprene sleeve 14a and the elongate band of wrap material are illustrated in phantom so as not to obstruct viewing of the loop structures. There are four loop structures shown in FIG. 4, two rear loop structures 20 and 24, and two front loop structures 30 and 34. Each of the loop structures comprises a plurality of individual loops interconnected to one another in overlapping manner in their longitudinal direction, which is also the longitudinal direction of the thigh. In the preferred embodiment the rear loop structure 20 has first, second and third loops 20a, 20b and 20c respectively as arranged from top to bottom. Similarly, rear loop structure 24 comprises three successively aligned and overlapping loops 24a, 24b and 24c respectively as arranged from top to bottom. Front loop structure 30 has upper and lower loops 30a and 30b, and front loop structure 34 has upper loop 34a and lower loop 34b. It will be appreciated that the number of loop structures and the number of loops within a loop structure can vary within the scope of this invention.

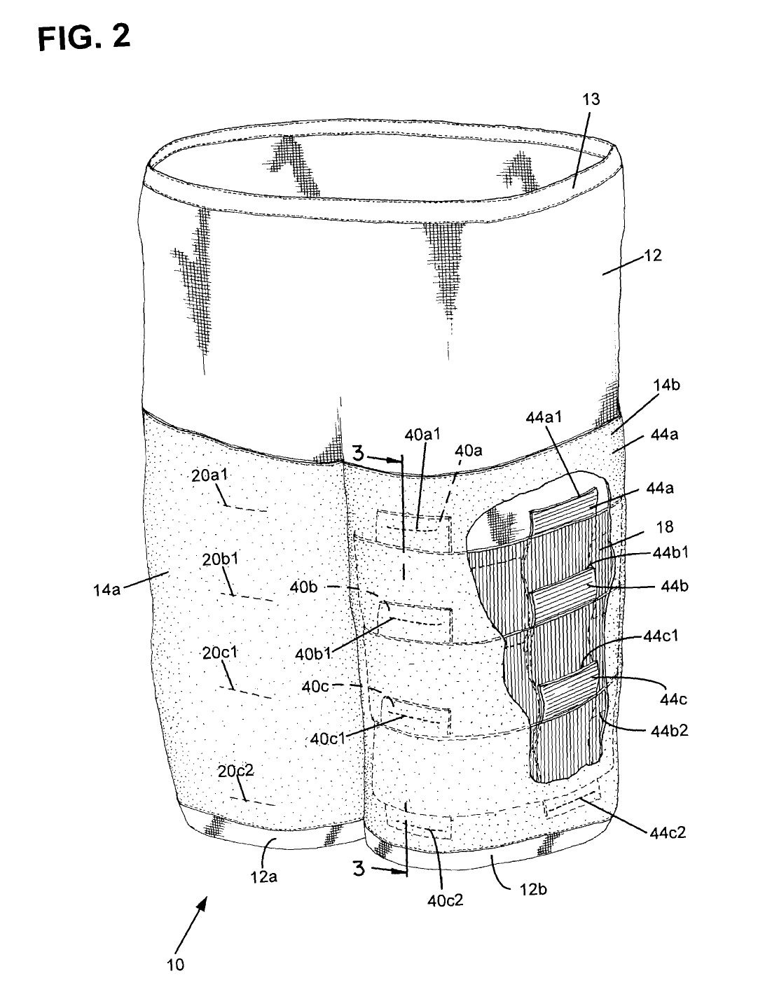

A mirror image set of loop structures as above-described for the left leg sleeve 14a is associated with the right leg sleeve 14b. Right leg loop structures as they would appear viewed from the front in similar manner as for the left leg descriptions of FIG. 4, would be respectively numbered 40a, 40b, 40c (right back loops from top to bottom), 44a, 44b, 44c (left back loops from top to bottom), 50a, 50b (left front loops from top to bottom), and 54a, 54b (right front loops from top to bottom). The left back loops 44 of the right leg of the garment are illustrated in FIG. 2, which is a figure that views the hamstring support garment from the back side of the garment when worn. The neoprene sleeve material 14b is shown broken away to illustrate the rear loops 44.

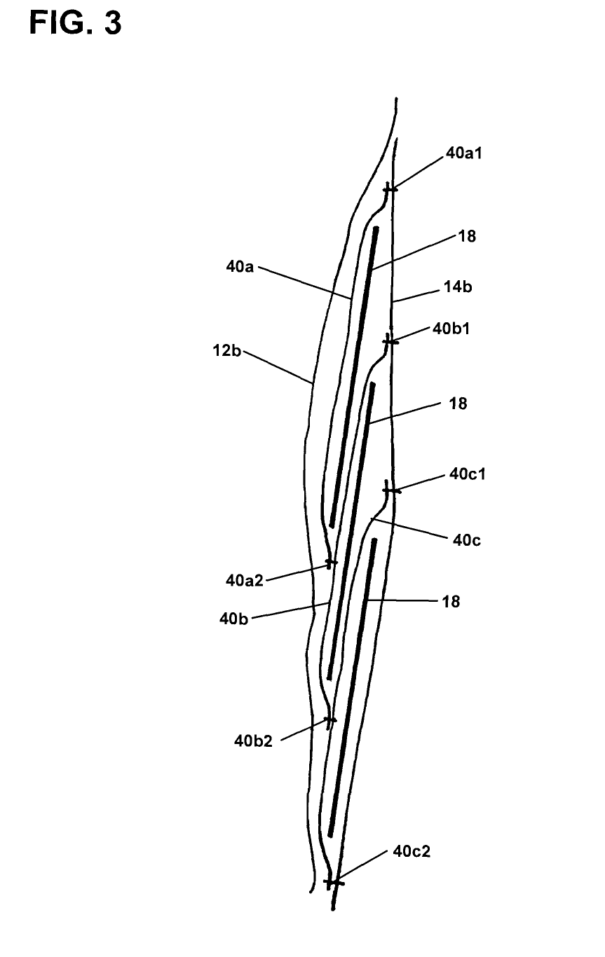

The individual loops of the loop structures are longitudinally sewn together and/or relative to one another in partially overlapping manner as shown in FIG. 3 for the right leg loop structure 40. Each of the loop structures is fixedly anchored to the inner surface of the neoprene sleeve of which it forms a part. In the preferred embodiment, the loop structures are anchored to the neoprene sleeves 14 by sewing. However, the loop structures could also be detachably secured to their respective sleeves by, for example, hook and loop fasteners (such as Velcro® fasteners) or the like.

The top end portion of each of the individual loops (20a-20c, 24a-24c, 30a-30b, 34a-34b, 40a-40c, 44a-44c, 50a-50b, 54a-54b) is secured to the inside surface of the sleeves 14. The bottom end portion of each of the lowermost loops (20c, 24c, 30b, 34b, 40c, 44c, 50b and 54b) of each of the loop structures is secured to the inner surface of its respective sleeve 14. The lower end portions of the upper two loops (20a, 20b, 24a, 24b, 40a, 40b and 44a, 44b) of the back, three-loop structures, and the lower end portions of the upper loops (30a, 34a, 50a and 54a) of the front, two-loop structures, are secured to the intermediate loop material of the next lower loop in their respective loop structures. In the preferred embodiment, the loop structures are not anchored directly to the compression pant material, which is free to move over and relative to the loop structures and the wrap band material supported thereby. This configuration is best shown in the cross-sectional view of the loop structure 40 in FIG. 3.

The loop anchoring stitches described above, when illustrated in the Figures, are numbered according to their respective loops as follows: (“loop number” “n”), where the “n” represents the position of the stitch on a loop. The numeral “1” represents the upper anchor stitch of the loop, and the numeral “2” represents the lower anchor stitch of the loop, (e.g. the upper and lower anchor stitches for loop 40a are 40a1 and 40a2 respectively). The loop anchor stitching pattern for the loop structure 40 is best shown in the cross-sectional depiction of FIG. 3. Referring thereto, upper end portions of loops 40a, 40b and 40c are all anchored to the neoprene sleeve 14b at 40a1, 40b1 and 40c1 respectively. The lower end portion of the bottom loop 40c is also anchored to the neoprene sleeve 14b at 40c2. The lower edge of the upper loop 40a overlaps the next lower loop 40b and is anchored at 40a2 to an intermediate portion of the second loop 40b. Similarly, the lower end portion of the middle loop 40b overlaps the bottom loop 40c and is anchored at 40b2 to an intermediate portion of the lower loop 40c.

A similar loop anchoring configuration is used for all of the other loop structures of the hamstring support garment. For example, referring to FIG. 4 which illustrates the loop structures 20, 24, 30 and 34 of the left leg of the garment, the anchor stitching descriptions are as follows. For the loops of rear loop structure 20, stitchings 20a1, 20b1, 20c1 and 20c2 are secured to the neoprene sleeve 14a. Anchor stitches 20a2 and 20b2 are respectively connected to intermediate portions of loops 20b and 20c respectively. For the loops of rear loop structure 24, stitches 24a1, 24b1, 24c1 and 24c2 are anchored to the neoprene sleeve 14a. Stitches 24a2 and 24b2 are anchored respectively to intermediate portions of the loops 24b and 24c respectively. For the loops of the front loop structure 30, the stitches 30a1, 30b1 and 30b2 are anchored to the neoprene sleeve 14a, and the stitch 30a2 is anchored to an intermediate portion of the lower loop 30b. For the loops of the front loop structure 34, stitchings 34a1, 34b1, and 34b2 are anchored to the neoprene sleeve 14a, and the stitching 34a2 is anchored to an intermediate portion of the loop 34b material. The loop anchor stitching configuration for the right leg sleeve 14b is similar to that illustrated in FIG. 4 with respect to the left leg, but would be a mirror image thereof as previously discussed.

In the preferred embodiment, the loop structures are preferably configured from a heavy nylon webbing material preferably about 1.25 inches wide and about 1/16 inches thick. Thicker loop materials facilitate support of the wrap band, and minimize binding of the wrap band material as it slides through or relative to the loops. Also in the preferred embodiment, the longitudinal length of the loops between anchor stitchings is about 3.5 inches long for accommodating a 3 inch wide wrap band (hereinafter discussed). In the preferred embodiment, the contiguous loops in a loop structure are configured to overlap one another from about 0.75 to 1.0 inches, or from between about 0.25 to about 0.33 percent of the wrap band width. For example, for the loop structure 20, the second loop 20b overlaps the first loop 20a by 0.75 to 1.0 inches. A similar overlap applies for the third loop 20c overlapping the second loop 20b.

The width of the loop material can vary. Wider loops, as opposed to string or shoelace width loops are preferred for providing better retaining support for the wrap band and comfort to the wearer. Preferably, the width of the loop material would range from about 1.0 inches to about 3.0 inches. Similarly, the length of a respective loop between its anchor points can vary depending upon the width of the wrap band material. The longitudinal length of a loop must be somewhat greater than the width of the wrap band material, to enable relatively free sliding motion of the band material through the retaining loops.

As described above, the loop structures are designed to slidably retainably guide a continuous band of wrap material therethrough. The wrap band for the left leg has been labeled in the figures at 17, and the wrap band for the right leg is labeled at 18. The wrap material comprises a stretchable elastive material such as four-way stretchable spandex material. In the preferred embodiment, the elastomeric wrap band is 3 inches wide and of a length sufficient to make three continuous overlapping revolutions about the thigh of the user. The loop structures of a leg portion of the hamstring support garment 10 are connected or anchored to the neoprene sleeve material so as to guide the elastomeric wrap strip in a downward spiral manner. The downward spiral configuration is attained by anchoring the successive loop structures through which the wrap band passes at successively lower longitudinal positions along the neoprene sleeve as illustrated, for example, in FIG. 4 with respect to the neoprene sleeve 14a.

An upper end 17a of the wrap band 17 is fixedly anchored by stitching or is detachably anchored, for example, by hook and loop-type fasteners, to the inside surface of the neoprene sleeve 14a. In the preferred embodiment illustrated, the first end 17a of the band 17 is anchored along the same stitching line as the 20a1 upper stitching of the first loop 20a of the loop structure 20. The band 17 is then folded at approximately a 45 degree angle at 17b and is threaded through the first loop 20a (from left to right, or in a clockwise direction as viewed from the top of the sleeve 14a) as illustrated by the arrow W, indicating the winding direction of the wrap 17. The upper stitching 24a1 of the upper loop 24a of the loop structure 24 is longitudinally positioned lower along the sleeve 14a than the stitching 20a1 such that the wrap 17 begins to spiral downwardly around the inner circumference of the sleeve 14a and around the outer circumference of the wearer's leg as the band 17 passes through the upper loop 24a along the back portion of the sleeve 14a. The upper stitching 30a1 of the upper front loop 30a is longitudinally positioned lower along the sleeve 14a than the stitching 24a1 such that the upper loop 30a receives the wrap band 17 along the downward spiral direction as illustrated in FIG. 4 as the wrap traverses along the front portion of the sleeve 14a. Similarly, the upper stitching 34a1 of the front upper loop 34a is positioned longitudinally lower than the stitching 30a1 so as to receive the wrap band 17 in its downwardly spiraling path. As the band passes through the upper loop 34a and again proceeds toward the back portion of the sleeve 14a to complete its first revolution around the sleeve or leg, it is next slidably received within the second loop 20b of the loop structure 20 at a position as previously described such that the upper edge 17c of the second revolution of the wrap band 17 overlaps the lower edge 17d of the first revolution portion of the wrap band 17. The overlap is determined and maintained by the overlapping loop structure as previously described. The band 17 then continues on in the downwardly spiraling manner as illustrated in FIG. 4 through the second loop 24b, through the lower front loop 30b and the lower front loop 34b to complete its second revolution. The third revolution begins by the wrap band 17 passing through the lowermost back loops 20c and 24c and back toward the front portion of the sleeve 14a.

A generally vertical longitudinal slot 55 is formed through the neoprene material of the sleeve 14a at the front outer portion of the sleeve 14a as viewed in FIGS. 1 and 4. After passing through the lowermost rear loop 24c, the wrap band 17 passes through the slot 55 in the front portion of the sleeve 14a and continues in its downward spiral rotation overlapping the second revolution wrap as illustrated in FIGS. 4 and 1. The second or free end 17e of the wrap band 17 is detachably secured to the outer surface of the sleeve 14a by an appropriate fastener such as a hook and loop fastener in a manner well-known in the art, and illustrated at 58 in FIG. 1. A similar downwardly spiraling overlapping configuration exists for the right leg sleeve 14b except that the spiral winding is in the opposite direction (i.e. counterclockwise when viewed from above as in FIGS. 1 or 2), such that the wrap band 18 of the sleeve 14b exits the slot 56 of the sleeve 14b along the left edge of the right leg (as viewed in FIG. 1), and the second end 18e of the band 18 is detachably secured by an appropriate fastener 59 to the outside surface of the sleeve 14b, as illustrated in FIG. 1.

To use the hamstring support garment of the present invention, the ends 17e and 18e of the wrap bands 17 and 18 are detached from their respective fasteners 58 and 59, allowing the wrap bands 17 and 18 to retractably slide into their respective sleeves through the slots 55 and 56. The wrap band ends 17e and 18e are then temporarily fastened back to their respective fasteners 58 and 59 in a loosened position so as to prevent the ends 17e and 18e from being retracted back into their slots 55, 56. This allows the compression pant material of the garment legs to radially expand to accommodate the user's legs. The user inserts his/her legs within the leg portions of the compression pant 12 and pulls the garment up to an operative position as illustrated in FIG. 1, with the upper band 13 of the garment disposed generally at the wearer's waist, and such that the support sleeves 14a and 14b are positioned around the thighs of the wearer. In such position, the wrap bands 17 and 18 loosely encircle the wearer's legs in downwardly spiraling manner as retained by their respective loop structures and are separated therefrom by the material of the underlying compression pant leg portions 12a and 12b. After longitudinally adjusting the leg portions of the compression pant 12 to the proper desired position along the thighs of the wearer, the elastive wrap bands 17 and 18 are tightened to provide the desired compressive support pressure around the thigh of the user. Each band is individually tightened by simply releasing the band ends 17e, 18e from their respective fasteners 58, 59 and longitudinally pulling the ends 17e, 18e to slide the bands of wrap material 17 and 18 through their respective slots 55 or 56 formed in the sleeves 14a or 14b. Pulling on the end of a wrap band causes the band to longitudinally move by sliding through its retaining loops thereby tightening the wrap around the wearer's leg, to apply radial inward pressure to the wearer's thigh and underlying hamstring muscles. There is enough tolerance in the support loops to provide relatively free sliding motion of the wrap bands therethrough. Further, the underlying compression pant leg portions 12a and 12b enable the wrap band material to slide thereover without directly engaging or irritating the wearer's skin. When the desired compressive force is attained, the user simply refastens the free ends 17e, 18e of the wrap bands to their respective fasteners 58 or 59 on the front surfaces of the sleeves to fix the desired compressive force applied to the thigh and underlying hamstring muscles. Should the user thereafter desire to either decrease or increase the compressive force on a leg, the user can rapidly adjust the pressure by releasing the second end 17e/18e of the wrap band from its fastener 58/59, and by either loosening or tightening pressure on the band by allowing some of the band to retract into the associated neoprene sleeve or by pulling more band out of the sleeve respectively.

The garment 10 may be worn as an external garment. However, the adjustment feature can readily be performed even if the user is wearing an overlying garment or sportswear item, by simply raising the bottom edge of the overlying garment to expose the adjustment end of the wrap band to be adjusted. Therefore, the user can quickly adjust the garment's compressive force on-the-fly, without undue interruption of the sport or activity in which he/she is engaged.

While the invention has been illustrated with reference to a preferred construction as applicable to treating hamstring muscles, and with reference to preferred materials, it will be understood that other constructions and materials can be used without departing from the spirit and intent of the invention. For example the two part compression pant and overlying sleeve construction could be replaced by a single pant structure that serves both of the functions of providing compressive pressure and anchoring of the loop structures for the wrap bands. Examples of alternative materials for the various garment components have been suggested above. Those skilled in the art may envision yet others, including materials yet to be commercialized. Those materials described in the preferred embodiment were selected both for their functionality and because they are currently readily commercially available.

Similarly other design variations can be made. For example, the direction of the wrap winding could be reversed, and could also wind from bottom up instead of from top down. It will be appreciated that while a preferred embodiment, description and application of the invention have been disclosed, other modifications of the invention not specifically disclosed or referred to herein will be apparent to those skilled in the art in light of the foregoing description. This disclosure is intended to provide a specific example of a preferred embodiment structure and application that clearly discloses an apparatus and method of the present invention and its operative principles. Accordingly, the invention is not limited to any particular embodiment or configuration or component parts thereof or to the use of any particular materials for their construction. All alternatives, modifications, and variations of the present invention which fall within the spirit and broad scope of the appended claims are covered.

I claim:

1. A muscle support garment comprising:
a. an outer garment configured in part to encircle a treatment portion of a person's body;
b. an elongate band of wrap material having first and second ends, wherein at least one of said ends is securable to said outer garment;
c. a retainer assembly configured to operatively mount a substantial portion of said band of wrap material inside of said outer garment, in spiral overlapping manner encircling said body treatment portion; and
d. a portion of said band of wrap material extending externally of said outer garment to enable length adjustment tensioning of said wrap material about said body treatment portion, from external of said outer garment.

2. The muscle support garment of claim 1, wherein said first end of said band of wrap material if fixedly secured to said outer garment.

3. The muscle support garment of claim 1, wherein at least one of said ends of said band of wrap material is secured to an inner surface of said outer garment.

4. The muscle support garment of claim 1, wherein at least one of said ends of said band of wrap material is adjustably securable to an outer surface of said outer garment.

5. The muscle support garment of claim 1, wherein said outer garment defines a passageway therethrough through which said band of wrap material is slidably entrained.

6. The muscle support garment of claim 5, wherein at least one of said first or second ends of said band of wrap material is detachably adjustably securable to the outer surface of the outer garment.

7. The muscle support garment of claim 1, wherein said outer garment is configured at least in part of neoprene material.

8. The muscle support garment of claim 7, wherein said band of wrap material is securable to said neoprene material.

9. The muscle support garment of claim 1, wherein said retainer assembly slidably mounts said band of wrap material for movement relative to said retainer assembly.

10. The muscle support garment of claim 9, wherein said retainer assembly comprises a plurality of loops within said outer garment through which said band of wrap material is entrained.

11. The muscle support garment of claim 10, wherein said plurality of loops are arranged in a plurality of overlapping columns of said loops.

12. The muscle support garment of claim 1 wherein said outer garment includes a liner material disposed to lie between said body treatment portion and said band of wrap material.

13. The muscle support garment of claim 12 wherein said liner material is disposed to lie between said body treatment portion and said retainer assembly.

14. A muscle support garment, comprising:
(a) a compression pant configured to extend generally between a wearer's waist and down toward a knee area and having at least one leg portion configured to encircle a wearer's thigh;
(b) a plurality of loop structures configured for attachment to the compression pant, said loop structures including a plurality of longitudinally overlapping loops; said loop structures being cooperatively mountable to said compression pant for continuously entraining a wrap band in overlapping spiral manner around the user's thigh;
(c) fasteners for securing said loop structures to said compression pant;
(d) an elongate wrap band configured and sized to slide within the loops of said loop structures and being anchored at its ends to said compression pant; and
(e) wherein said loop structures orient said wrap band in overlapping spiral wrap manner about the thigh of a user; providing radial compressive force to the thigh and underlying muscles.

15. The muscle support garment of claim 14, wherein said fasteners anchor said loop structures to the inner surface of said compression pant that addresses the thigh.

16. The muscle support garment of claim 14, wherein a first end of said elongate wrap band is anchored to an inner surface of said support garment and an opposite longitudinally disposed second end of said wrap band is anchored to an outside surface of said garment.

17. The muscle support garment as recited in claim 16, wherein the garment includes an egress port through which said wrap band passes from the inside to outside surfaces of said garment.

18. The muscle support garment of claim 16, wherein said second end of said wrap band is detachably anchored to the outside surface of said garment.

19. The muscle support garment of claim 14, wherein at least a part of said one leg portion comprises neoprene material.

20. The muscle support garment of claim 19, wherein said fasteners anchor said loop structures to said neoprene material.

21. The muscle support garment of claim 19, wherein at least a part of said compression pant comprises elastive material.

22. The muscle support garment of claim 21, wherein at least a part of said compression pant comprises condensation polymer material.

23. The muscle support garment of claim 14, wherein the material of said loop structures at least in part comprise nylon.

24. The muscle support garment of claim 14, wherein at least some of said loops of the loop structures are at least 0.75 inches in width.

25. The muscle support garment of claim 14, wherein said elongate wrap band comprises a stretchable material.

26. The muscle support garment of claim 19, wherein said one leg portion comprises a layer of lining material configured to lie between a wearer's leg and the neoprene material.

27. The muscle support garment of claim 26, wherein said fasteners anchor said loop structures to said neoprene material, and wherein said lining material is disposed between said loop structures and said wearer's leg.

28. A method of applying a compressive wrap to a thigh portion of a person comprising the steps of:
(a) providing a compression pant having at least one leg portion for surrounding the thigh portion;
(b) securing a longitudinally overlapping loop arrangement to the inside of the leg portion;
(c) entraining an elongate wrap band through a plurality of loops of said loop arrangement such that said wrap band encircles the wearer's thigh portion in overlapping spiraling manner;
(d) longitudinally pulling on said wrap band to apply radial compressive force to the thigh portion; and
(e) securing the ends of the wrap band to the leg portion to maintain a desired compressive force against the thigh portion.

29. The method of claim 28, including adjusting the desired compressive force by:
(a) detaching one end of the wrap band from the leg portion;
(b) causing the wrap band to longitudinally move and slide within said loop arrangement to change the compressive force applied to the thigh portion; and
(c) refastening the one end of the wrap band to the leg portion to maintain the changed compressive force.

30. The method as recited in claim 29, wherein the step of detaching one end of the wrap band is performed external of said leg portion.

31. The method of claim 28, including the step of providing a liner to the leg portion configured to lie adjacent the thigh portion and between the thigh portion and loop arrangement.

32. A hamstring muscle support garment comprising:
(a) a compression pant material configured to extend from a waist portion toward a knee of a wearer and having at least one downwardly depending leg portion, said compression pant comprising a blend of condensation polymer and elastive materials;
(b) an external sleeve of material secured along at least one of its upper or lower end portions to said leg portion of the compression pant material;
(c) a loop structure having a plurality of loops secured to an inner surface of said external sleeve and addressing an outer surface of said compression pant material;
(d) an elongate band of wrap material slidably entrained through said loops and oriented in an overlapping spiral configuration about the inside of said leg portion; and
(e) at least one fastener selectively securable along the length of said wrap material, wherein the operative length of said wrap material within said leg portion can be changed, thereby varying compressive forces applied by the wrap to a thigh of a wearer of the garment.

33. A hamstring support garment comprising:
(a) a compression pant having at least one leg portion;
(b) an elongate wrap band; and
(c) wrap retaining apparatus configured to operatively connect said elongate wrap band to said compression pant and to align said elongate wrap band along an inner circumferential surface of said one leg portion, in overlapping spiraling manner, to encircle a thigh portion of a wearer of said garment.

34. The hamstring support garment of claim 33, wherein said wrap retaining apparatus is configured to allow longitudinal sliding motion of the wrap band relative to the retaining apparatus.

35. The hamstring support garment of claim 33, wherein said elongate wrap band comprises an elastive stretchable material.

36. The hamstring support garment of claim 33, wherein said retaining apparatus comprises a retaining loop apparatus.

37. The hamstring support garment of claim 36, wherein said retaining loop apparatus is configured to attach to said inner circumferential surface of said leg portion.
